(19) United States
(12) Patent Application Publication
(10) Pub. No.: US 2003/0207854 A1
(43) Pub. Date: Nov. 6, 2003

(54) Antiprogestins with partial agonist activity

(76) Inventor: S. Stoney Simons, Jr., Bethesda, MD (US)

Correspondence Address: NEEDLE & ROSENBERG, P.C., SUITE 1000, 999 PEACHTREE STREET, ATLANTA, GA, 30309-3915, US

(21) Appl. No.: 10/239,548

(22) PCT Filed: Mar. 23, 2001
(86) PCT No.: PCT/US01/09395

(51) Int. Cl. A61K 31/573

(52) U.S. Cl. 514/179

(57) ABSTRACT

The present invention features methods of inhibiting progestin activity in a cell or in a subject in need of such inhibition, and methods of treating or preventing progestin-dependent conditions in a subject in need of such treatment or prevention. The methods include administering an effective amount of a C-17-derivatized dexamethasone antiprogestin to the cell or the subject. The invention also provides methods of screening substances for progestin or antiprogestin activity, and methods of detecting progestins, progestin agonists, or antiprogestins in a sample, using the C-17-derivatized dexamethasone antiprogestins of the invention.

### ACKNOWLEDGMENTS

This invention was made with intramural support from the National Institutes of Health. The government has certain rights in the invention.

### FIELD OF THE INVENTION

This invention relates generally to the identification of a class of compounds that behave as antiprogestins with partial agonist activity, and methods for their use.

### BACKGROUND OF THE INVENTION

A primary effect of steroid hormones is to regulate the rates of transcription of responsive genes during development, differentiation, and homeostasis. The basic steps appear to be the same for all members of the steroid/nuclear receptor superfamily that bind ligands. After a ligand enters a cell and binds to its cognate intracellular receptor protein, the resulting receptor-ligand complex undergoes activation to acquire an increased affinity for DNA. This receptor-ligand complex then attaches to specific, biologically active DNA sequences, called hormone response elements (HREs) and, in association with various cofactors, is thought to interact with the transcriptional machinery to modify the rates of transcription of the target gene.

The human progesterone receptor (PR) occurs as three different isoforms: PR-A, PR-B, and PR-C (Kastner et al., EMBO J 9:1603-1614, 1990; Wei et al., Mol Endo 10:1379-1387, 1996), of which PR-A and PR-B are the most abundant. However, the ratio of PR-A vs. PR-B isoforms is not constant among target tissues, and this can alter the cellular response, because the activity of each isoform can vary.

Antisteroids are defined as compounds that block the action of agonist steroids. However, virtually all antisteroids retain some agonist activity under selected conditions. Partial agonist activity was once viewed as a defect of an antisteroid when the objective was to synthesize compounds that would block all of the activity of an agonist steroid. More recently, it has become clear that partial agonist activity can be a highly desirable feature. Thus, in endocrine therapies of disease states, it is beneficial to retain agonist activity in as many of the unaffected tissues as possible. For example, tamoxifen is now widely used as an antiestrogen in the treatment of breast cancer, because tamoxifen behaves as an estrogen in bone tissue, and therefore, does not demineralize the bones of patients.

Although most steroid receptors each bind several ligands that display partial agonist activity, there are very few compounds that exhibit partial progestin activity under a wide variety of conditions. RU-486, the most commonly used antiprogestin, displays partial agonist activity only under selected conditions (Meyer et al., EMBO J 9:3923-3932, 1990; Jackson et al. Mol Endo 11:693-705, 1997). A closely related derivative of RU-486, known as RTI 3021-020 (RTI-020), has been described as a partial agonist in T47D human breast carcinoma cells. However, this compound is not active in CV-1 monkey kidney cells (Wagner et al., Proc Natl Acad Sci USA 93:8739-8744, 1996), which are used for studies on the mechanism of PR action.

Antiprogestin compounds with partial agonist activity are useful for treating various progestin-regulated diseases and conditions. Such antiprogestin compounds also have a large number of research applications. However, the few known antiprogestins have only limited partial agonist activity, and there remains a need in the art for antiprogestins with broad-range partial agonist activity. The present invention overcomes previous shortcomings in the art by providing methods for inhibiting progestin activity, based on the identification of a class of compounds that have a broader range of progestin partial agonist activity than previously known antiprogestin compounds.

### SUMMARY OF THE INVENTION

In a first aspect, the invention features a method of inhibiting progestin activity in a subject in need of such inhibition. The method includes administering an effective amount of a C-17-derivatized dexamethasone antiprogestin to the subject.

In a second aspect, the invention features a method of treating or preventing a progestin-dependent condition in a subject in need of such treatment or prevention. The method includes administering an effective amount of a C-17-derivatized dexamethasone antiprogestin to the subject.

In preferred embodiments of the first and second aspects of the invention, the subject may be a mammal, such as a human, a farm animal, a domestic animal, or a laboratory animal, and the subject may be female.

In various other embodiments of the first and second aspects of the invention, the method may be used for: regulating menses; treating or preventing a benign progestin-dependent condition, such as endometriosis, leiomyoma, ovarian cysts, premenstrual syndrome, anemia, dysmenorrhea, or pelvic inflammatory disease; or treating a progestin-responsive tumor, such as a breast carcinoma, an ovarian carcinoma, a prostate carcinoma, an endometrial carcinoma, a cervical carcinoma, a leiomyosarcoma, or a meningioma.

In other preferred embodiments of the first and second aspects of the invention, the method may be used for preventing pregnancy. For example, the C-17-derivatized dexamethasone antiprogestin may be administered prior to ovulation or prior to coitus, or after ovulation or after coitus. In still other embodiments of the first and second aspects of the invention, the administering inhibits implantation of an embryo in the subject or the administering inhibits ovulation in the subject.

In yet other embodiments of the first and second aspects of the invention, the method is for inducing cervical ripening in a female. The cervical ripening may be preparatory to labor and delivery of offspring, or preparatory to dilatation and curettage. In yet another embodiment of the first and second aspects of the invention, the administering is carried out in order to induce expulsion of an embryo or fetus from the subject.

In a third aspect, the invention features a method of detecting a progestin or a progestin agonist in a sample. The method includes the steps of: a) contacting the sample with a reaction mixture containing a progesterone receptor and a C-17-derivatized dexamethasone antiprogestin; and b) measuring progestin activity in the reaction mixture, wherein an increase in progestin activity, as compared to the amount of progestin activity measured in a reaction mixture not contacted with the sample, detects a progestin or a progestin agonist in the sample.

In a fourth aspect, the invention features a method of screening a substance for progestin activity. The method includes the steps of: a) contacting the substance with a reaction mixture including a progesterone receptor and a C-17-derivatized dexamethasone antiprogestin; and b) measuring progestin activity in the reaction mixture, wherein an increase in progestin activity, as compared to the amount of progestin activity measured in a reaction mixture not contacted with the substance, identifies a substance having progestin activity.

In a fifth aspect, the invention features a method of screening a substance for antiprogestin activity. The method includes the steps of: a) contacting the substance with a first reaction mixture including a progesterone receptor and a progestin or a progestin agonist; b) measuring progestin activity in the first reaction mixture; and c) comparing the progestin activity in the first reaction mixture with an amount of progestin activity in a second reaction mixture including a progesterone receptor, a progestin or a progestin agonist, and a C17-derivatized dexamethasone antiprogestin. A decrease in progestin activity in the first reaction mixture that is at least about 20% of the decrease in progestin activity in the second reaction mixture, compared to the amount of progestin activity in the second reaction mixture in the absence of the antiprogestin, identifies a substance having antiprogestin activity.

In a sixth aspect, the invention features a method of detecting an antiprogestin in a sample. The method includes the steps of: a) contacting the sample with a first reaction mixture comprising a progesterone receptor and a progestin or a progestin agonist; b) measuring progestin activity in the first reaction mixture; and c) comparing the progestin activity in the first reaction mixture with an amount of progestin activity in a second reaction mixture including a progesterone receptor, a progestin or a progestin agonist, and a C-17-derivatized dexamethasone antiprogestin. A decrease in progestin activity in the first reaction mixture that is at least about 20% of the decrease in progestin activity in the second reaction mixture, compared to the amount of progestin activity in the second reaction mixture in the absence of the antiprogestin, detects an antiprogestin in the sample.

In a seventh aspect, the invention features a method of inhibiting progestin activity in a cell in need of such inhibition. The method includes administering an effective amount of a C-17-derivatized dexamethasone antiprogestin to the cell.

In preferred embodiments of the seventh aspect of the invention, the cell may be a mammalian cell, such as a human, monkey, mouse, or rat cell. The cell may be a primary cell, i.e., taken directly from the mammal, or the cell may be from an established cell line, for example, CV-1 or 1470.2.

In an eighth aspect, the invention features a method of detecting a gene whose 20 expression is modulated by an antiprogestin. The method includes the steps of: a) contacting a progesterone receptor with a C17-derivatized dexamethasone antiprogestin, wherein the progesterone receptor is present within a sample that includes a gene that is positively or negatively regulated by the progesterone receptor; b) measuring the expression level of the gene in the sample; and c) comparing the expression level of the gene in the sample with the expression level of the gene in a sample not contacted with the C17-derivatized dexamethasone antiprogestin. An increase or decrease in the expression level of the gene in the sample contacted with the C17-derivatized dexamethasone antiprogestin, compared to the expression level of the gene in the sample not contacted with the C-17-derivatized dexamethasone antiprogestin, detects a gene whose expression is modulated by an antiprogestin.

In a preferred embodiment of the eighth aspect of the invention, the C-17-derivatized dexamethasone antiprogestin may be a partial antiprogestin, i.e., a partial progestin agonist. In another embodiment of the eighth aspect of the invention, the sample not contacted with the C-17-derivatized dexamethasone antiprogestin may be contacted with a pure progestin agonist, such as R5020.

In still other embodiments of the eighth aspect of the invention, a progestin or a progestin agonist may be added to the sample, for example, before, after, or at the same time as the C17-derivatized dexamethasone antiprogestin is added to the sample. In yet other embodiments of the eighth aspect of the invention, the sample may be a mammal, a cell, or a cell extract.

In preferred embodiments of all of the above aspects of the invention, the C-17-derivatized dexamethasone antiprogestin may be dexamethasone-21-mesylate or dexamethasone-oxetanone.

### BRIEF DESCRIPTION OF THE DRAWINGS

FIG. 1 is a graph showing the effect of PR concentration on total transactivation and -fold induction of a progestin-responsive reporter gene in transiently co-transfected 1470.2 cells treated with R5020.

FIG. 2 is a diagram showing the structures of glucocorticoid agonists and progestin agonists, partial agonists and antagonists.

FIG. 3 is a graph showing the Dex-Mes-induced transcriptional activity of a progestin-responsive reporter gene in 1470.2 cells co-transfected with the reporter gene and PR cDNA.

FIG. 4 is a graph showing the Dex-Ox-induced transcriptional activity of a progestin-responsive reporter gene in 1470.2 cells co-transfected with the reporter gene and PR cDNA.

FIG. 5A is a graph showing the relative activity of Dex, Dex-Mes, Dex-Ox, and RTI 3021-020 with the glucocorticoid receptor in 1470.2 cells not transfected with PR cDNA.

FIG. 5B is a graph showing the relative partial progestin activity of Dex, Dex-Mes, Dex-Ox, and RTI 3021-020 in 1470.2 cells transfected with different concentrations of PR cDNA.

FIG. 6 is a graph showing the cell-free competition of [³H]R5020 binding to PR by non-radioactive steroids.

FIG. 7 is a graph showing the antiprogestin properties of Dex-Mes and Dex-Ox.

### DETAILED DESCRIPTION OF THE INVENTION

Dexamethasone (Dex) is a potent glucocorticoid that displays very low affinity (Ojasoo et al., J Med Chem 31:1160-1169, 1988) and activity (Vegeto et al., Mol Endocrinol 7:1244-1255, 1993) for the progesterone receptor (PR). The present invention provides the surprising discovery that C-17 derivatives of Dex behave as antiprogestins with partial agonist activity. In particular, both Dex-21-mesylate (Dex-Mes), which is an affinity label and antagonist for glucocorticoid receptors (GR) (Simons and Thompson, Proc Natl Acad Sci USA 78:3541-3545, 1981), and Dex-oxetanone (Dex-Ox), which is a reversible antiglucocorticoid (Pons and Simons, J Org Chem 46:3262-3264, 1981; Lamontagne et al., Endocrinology 114:2252-2263, 1984), displayed high amounts of partial progestin activity with both PR-A and PR-B in two different cell lines, including CV-1 cells. Both Dex-Ox and Dex-Mes had affinities for the cell-free PR that were consistent with their whole cell action arising from binding to the intracellular PR protein, and displayed partial progestin activity under conditions where other reported partial progestins were inactive. In particular, both Dex derivatives were more active than either RU 486 or RTI 3021-020 under comparable conditions. These results are particularly significant, as very few partial agonists exist for PR. Furthermore, Dex-Mes and Dex-Ox retained partial agonist activity independent of the PR isoform (PR-A or PR-B), the enhancer, the promoter, the reporter, or the cells examined. Therefore, Dex-Mes and Dex-Ox offer promise as mixed progestin agonists under a wider variety of laboratory and clinical settings than the previously reported compounds.

The activity of Dex-Mes and Dex-Ox with PR was totally unexpected. Dex itself has very little, if any, affinity for, or activity with, PR. Thus, it would have been predicted that derivatives of Dex would have been similarly inactive. Clearly this is not the case, as Dex-Mes and Dex-Ox displayed between 5% and 95% agonist activity with PR, depending upon the conditions. These results suggest that other D-ring derivatives of Dex should be similarly active as partial PR agonists.

The significant affinity of Dex-Ox and Dex-Mes for PR, and appreciable partial progestin activity of these compounds in a wide variety of settings, both suggest that Dex-Ox and Dex-Mes should be particularly useful steroids in a variety of clinical and research applications. In contrast to Dex-Ox, Dex-Mes is an electrophilic affinity label that forms covalent bonds with the glucocorticoid receptor, although it does not covalently label PR (Simons et al., J Biol Chem 262:9676-9680, 1987). It should be appreciated that while both Dex-Ox and Dex-Mes are antiglucocorticoids, use of these compounds as antiprogestins will probably not have major side effects of blocking glucocorticoid action, as Dex-Ox and Dex-Mes usually display significant amounts of partial glucocorticoid activity (Mercier et al., J Steroid Biochem 25:11-20, 1986; Szapary et al., J Biol Chem 271:30576-30582, 1996). This is in contrast to the most commonly used antiprogestin, RU 486, which is a pure antiglucocorticoid in a wide variety of cells. Accordingly, the use of Dex-Mes and Dex-Ox as antiprogestins should be accompanied by reduced side effects, since fewer actions of glucocorticoid receptors will also be inhibited. Therefore, C-17 derivatives of Dex, such as Dex-Mes and Dex-Ox, offer the prospect of being useful partial progestin agonists in a broad variety of clinical and research situations in which partial progestin activity is desired.

Definitions

In this specification and in the claims that follow, reference is made to a number of terms that shall be defined to have the following meanings.

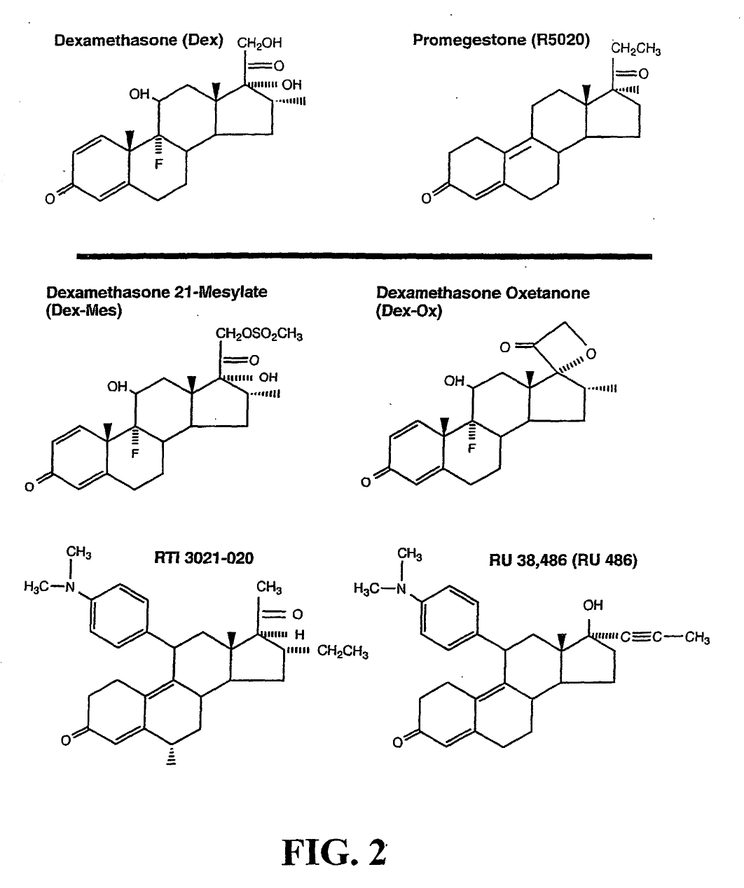

By “progestin” or “progestin agonist” is meant any compound, natural or synthetic, that is capable of binding to a progesterone receptor and stimulating progestin activity under physiological conditions. Examples of a progestin and a progestin agonist include, respectively, progesterone and promegestone (R5020; FIG. 2).

By “progestin activity” is meant the ability of progestins and progestin agonists to regulate certain physiological processes. The amount of progestin activity in a sample (e.g., an animal; tissue, blood, or urine from an animal; cells; or cell extracts) may be determined, for example, by measuring the amount of: progestin (or progestin agonist) binding to a progesterone receptor (PR), e.g., the PR-A, PR-B, or PR-C receptor; progestin-stimulated expression of a progesterone receptor-regulated reporter gene in a whole cell or in a cell-free transcription system; progestin-stimulated cell growth or gene expression; endometrial wall thickening during the secretory phase of the menstrual cycle; ovulation; fertility (e.g., as indicated by blastocyst implantation or parturition); and/or inhibition of cervical ripening prior to parturition. Relative progestin biological activity in a sample may be determined by comparing the amount of progestin activity within the sample to the amount of progestin activity of an appropriate control sample displaying a known amount of progestin activity.

By “increase in progestin activity” is meant a rise in the level of progestin activity measured as described above, for example, an augmentation of the amount of progestin bound to a progesterone receptor; an augmentation of the progestin-stimulated expression of a progesterone receptor-regulated reporter gene in a whole cell or a cell-free transcription system; or an acceleration of a progestin-dependent physiological process, such as cell growth. Preferably, the increase is by at least 1.5-fold to 2-fold, more preferably by at least 3-fold, and most preferably by at least 5-fold.

By “decrease in progestin activity” is meant a fall in the level of progestin activity measured as described above, for example, a reduction of the amount of progestin bound to a progesterone receptor; a reduction of the progestin-stimulated expression of a progesterone receptor-regulated reporter gene in a whole cell or a cell-free transcription system; or the slowing of a progestin-dependent physiological process, such as cell growth. Preferably, the decrease is by at least about 20%, and may be larger, e.g., by at least about 21%-40%, 41%-60%, 61%-80%, 81%-90%, or 91%-100%. A pure antiprogestin will cause a 100% decrease, but smaller decreases are often desirable in compounds that would be considered as partial antiprogestins (i.e., antiprogestins with partial agonist activity).

By “modulate” is meant to alter, either by increase or decrease.

By “progestin-dependent disease” or “progestin-dependent condition” is meant an affliction, disorder, or physiological state that depends upon progestin activity for its existence or that is exacerbated by progestin activity. Examples of progestin-dependent diseases include, for example, progestin-responsive tumors (as defined below); endometriosis; dysmenorrhea; and premenstrual syndrome. Examples of progestin-dependent conditions include, for example, ovulation; the presence of a secretory endometrium capable of supporting blastocyst implantation; the inhibition of uterine contraction and/or cervical ripening; and pregnancy.

By “progestin-responsive tumor” is meant a tumor that contains progesterone receptors and whose growth and/or metastatic potential is stimulated by the binding of progestin to progesterone receptors within the tumor.

By “antiprogestin” or “progestin antagonist” is meant any compound, natural or synthetic, that inhibits the activity of a progestin or a progestin agonist. A previously known example of an antiprogestin is RU 38,486 (RU 486; FIG. 2). The antiprogestins of the invention are C-17 derivatives of dexamethasone, e.g., as shown in FIG. 2 and as described herein. An antiprogestin or progestin antagonist may have (but does not necessarily have) progestin partial agonist activity.

By “C-17-derivatized dexamethasone” is meant a chemical derivative of dexamethasone that contains a chemical group at its C-17 position that is different from the chemical group shown at the C-17 position of dexamethasone (FIG. 2). As used herein, any C-17-derivatized dexamethasone will also have antiprogestin activity. Preferred examples of C-17-derivatized dexamethasones include dexamethasone 21-mesylate (Dex-Mes; FIG. 2) and dexamethasone oxetanone (Dex-Ox; FIG. 2).

By “progestin partial agonist” or “progestin mixed agonist” is meant an antiprogestin that, in certain circumstances (for example, in certain types of cells), displays some progestin activity. For example, a progestin partial agonist or mixed agonist may bind to and inhibit the ability of a progesterone receptor to activate expression of a progestin-dependent gene in one cell type, but, in binding to a progesterone receptor in a second cell type, may allow the progesterone receptor to activate expression of a progestin-dependent gene (for example, a gene encoding a progesterone receptor, a gene encoding α-lactalbumin, or a gene under the transcriptional regulation of a mouse mammary tumor virus (MMTV) promoter). This is analogous to the estrogen partial agonist or estrogen mixed agonist activity of tamoxifen, which behaves as an anti-estrogen in breast cancer cells, and as an estrogen in bone cells.

By “sample” is meant an animal (i.e., a warm-blooded animal, e.g., a human, a farm animal, a domestic animal, or a laboratory animal, as described hereinbelow); any body fluid (e.g., but not limited to, blood, urine, cerebrospinal fluid, semen, sputum, saliva, tears, joint fluids, body cavity fluids, or washings), tissue, or organ obtained from an animal; a cell (either within an animal, taken directly from an animal, or a cell maintained in culture or from a cultured cell line); a lysate (or lysate fraction) or extract derived from a cell; a molecule derived from a cell or cellular material (such as a DNA molecule used as a transcription template in a cell-free transcription assay); or a compound to be tested for progestin or antiprogestin activity (e.g., a test compound), which is assayed or analyzed for progestin or antiprogestin activity according to the methods of the invention.

By “modulation in the gene expression level” or “alteration in the level of gene expression” is meant a change in the amount of transcription, translation, mRNA stability, or protein stability, such that the overall amount of a product of the gene, i.e., an mRNA or polypeptide, is increased or decreased.

By “subject” is meant any animal that has progesterone receptors, to which the C17-derivatized dexamethasone antiprogestins of the invention are administered for therapeutic or experimental purposes, or to regulate fertility. For example, the subject may be a cold-blooded animal, such as a fish, a reptile, or an amphibian, or the subject may be a a warm-blooded animal, such as a human, a farm animal, a domestic animal, or a laboratory animal, as described herein. The subject may also be a cell or a DNA molecule in a cell-free environment (for example, a DNA molecule used as a transcription template in a cell-free transcription assay).

By “reaction mixture” is meant any environment that contains a progesterone receptor and can be used to measure progestin or antiprogestin activity of a test substance or progestin or antiprogestin activity within a sample. The reaction mixture may be, for example, within a test tube or within a well of a tissue culture dish or microtiter plate containing a progesterone receptor either in soluble form or bound to a solid phase; upon the surface of a filter or a polymer bead carrying a progesterone receptor; or within an animal, animal tissue, cell, cell lysate, or cell extract containing a progesterone receptor.

By “an effective amount” of a C-17-derivatized antiprogestin is meant an amount that is useful for performing the stated function (e.g., inhibition of progestin activity; treatment or prevention of a progestin-dependent condition) of the antiprogestin for which an effective amount is expressed. As will be described below, the exact amount required will vary, depending upon recognized variables, such as the subject to be treated, the reason for treatment, and the specific C17-derivatized dexamethasone antiprogestin compound employed. Thus, it is not possible to specify an exact “effective amount”. However, as described below, an appropriate “effective amount may be determined by one of ordinary skill in the art using only routine experimentation.

By “in need of” is meant a subject, such as a human, a farm animal, a domestic animal, or a laboratory animal, as described hereinbelow, or a cell, a cell-free transcription system, or a DNA molecule, for which it is desirable to treat, prevent, inhibit, or regulate a progestin-dependent disease or condition (or modulate progestin-dependent gene expression), or that is subjected to experimental administration of the C17-derivatized antiprogestins of the invention in order to study their pharmacological properties, such as, but not limited to, safety, efficacy, or physiological effects.

By “treat” is meant to administer the antiprogestin compounds of the invention to a subject, to a cell, or to a cell-free transcription system, in order to: eliminate a progestin-dependent disease or condition within a subject; stabilize or delay the progression of a progestin-dependent disease or condition within a subject; decrease the frequency or severity of symptoms and/or recurrences of a progestin-dependent disease or condition within a subject; or modulate progestin-dependent gene expression in the subject, cell, or cell-free transcription system.

By “prevent” is meant to minimize the chance that a subject will develop a progestin-dependent disease or condition, or to delay the development of a progestin-dependent disease or condition in a subject. For example, the antiprogestin compounds of the invention may be administered to minimize the chance that a female subject will become pregnant. For subjects belonging to families having hereditary progestin-dependent diseases and conditions, such as progestin-dependent breast cancer, progestin-dependent prostate cancer, progestin-dependent meningioma, endometriosis, or uterine fibroid tumors, for example, antiprogestin therapy may be initiated prior to disease onset, thereby lessening the chance that the subject will fall prey to the disease, and/or delaying the onset of the disease, relative to the time that onset would have occurred, had antiprogestin therapy not been initiated.

By “about” is meant ±10% of a recited value.

Therapeutic Uses of Antiprogestins

Progesterone and other progestins play a major role in reproductive health and function, as well as in other extra-reproductive physiological processes. Based upon our current knowledge of these various biochemical pathways, it is now considered desirable to employ antiprogestin therapy, when possible, for the treatment, prevention, or regulation of a broad array of progestin-dependent diseases and conditions. Compounds having antiprogestin activity are characterized by antagonizing the effects of progesterone or another progestin. As such, they may be used in any instance in which it may be desirable to modulate progestin activity.

For example, the C-17-derivatized dexamethasone antiprogestins of the invention may be used to regulate fertility during the female reproductive cycle of an animal of this invention. They may therefore be used to control irregularities in the human menstrual cycle or to synchronize or repress the fertile periods of commercial animals (e.g., fish, cows, sheep, pigs, goats, or chickens), laboratory animals (e.g., apes, chimpanzees, rats, mice, or guinea pigs), or domestic animals (dogs, cats, ferrets, birds, rabbits, or horses). They may also be used to lessen menstrual flow, thereby being useful in the treatment or prevention of anemia and dysmenorrhea. Regulation of the female reproductive cycle by the antiprogestins of the invention also provides useful therapy for the prevention or treatment of premenstrual syndrome, endometriosis, leiomyoma (uterine fibroids), and/or ovarian cysts. In addition, in a subject that has or has previously had pelvic inflammatory disease, in which scarring of the fallopian tubes may have occurred, the progestins of the invention may be used to lessen the chance that a tubal pregnancy may occur.

The C17-derivatized dexamethasone antiprogestins of the invention may be used as contraceptive agents for the prevention of pregnancy. Depending upon the desired dosage level and frequency, they may be administered, for example, on a daily, weekly, bi-weekly, or monthly basis, to inhibit ovulation and/or to inhibit implantation of a fertilized egg (or blastocyst) into the endometrial lining. Accordingly, the antiprogestins of the invention may be administered prior to ovulation, or subsequent to ovulation. In addition, the antiprogestins of the invention may be administered prior to coitus or after coitus to prevent pregnancy. An antiprogestin dosage that is effective for contraception is one that inhibits fertility, e.g., by inhibiting ovulation or implantation of a fertilized egg or blastocyst. Based on what is known in the art regarding progestin fluxes during the menstrual cycle and the role of progestin in ovulation and development of the secretory endometrium (i.e., an endometrium that allows implantation of a blastocyst), the skilled practitioner can readily ascertain an effective antiprogestin dosage level and frequency to be used in the methods of the invention. See, for example, Bygdeman et al., Eur J Contracept Reprod Health Care 4:103-107, 1999; Spitz and Robbins, Hum Reprod Update 4:584-593, 1998; Grow et al., Fertil Steril 69:936-943, 1998; and Zelinski-Wooten et al., Hum Reprod 13:2132-2138, 1998.

Antiprogestins may also be used for cervical ripening prior to labor induction, such as in term or post-tern pregnancies, or when labor must be induced to expel a dead fetus. Antiprogestins may also be used to end a pregnancy by inducing expulsion of a living embryo or fetus from the womb, or to promote dilatation of the cervix prior to uterine curettage. These uses are described, for example, in Mackenzie, Ann Acad Med Singapore 22:151-157, 1993; Sitruk-Ware et al., Contraception 41:221-243, 1990; and Hingorani et al., Acta Obstet Gynecol Scand Suppl 149:25-29, 1989.

Many types of tumors, for example, carcinomas of the breast, ovary, prostate, endometrium, and cervix, as well as leiomyosarcomas and meningiomas, have been shown to contain progestin receptors. These tumors are progestin-responsive, i.e., grow when exposed to progestins. Conversely, the growth of such progestin-responsive tumors is inhibited by the use of antiprogestin compounds. Therefore, the antiprogestins of the invention may be used to treat progestin-responsive tumors, whether benign or metastatic, or to prevent the initial development or recurrence of cancer, particularly in subjects known to be at greater than normal risk for such cancers, compared to the general population (e.g., subjects that have a hereditary risk for such cancers, or who have previously had cancer). The growth inhibitory effects of antiprogestins on progestin-responsive tumors are described further, for example, in Etreby and Liang, Breast Cancer Res Treat 49:109-117, 1998; Klijn et al., Hum Reprod 9 Suppl 1:181-189, 1994; Bakker et al., J Steroid Biochem Mol Biol 37:789-794, 1990; and Rose and Barnea, Oncogene 12:999-1003, 1996.

Additional information regarding the use (e.g., dosage, frequency, and physical effects) of antiprogestins for contraception, uterine ripening, expulsion of an embryo or fetus, treatment of uterine leiomyomas (fibroids), endometriosis, and other conditions associated with the female reproductive cycle, and treatment of various cancers may be found, for example, in U.S. Pat. No. 6,015,805; and Jang and Benet, J. Pharmacokinet Biopharm 25:647-672, 1997.

Methods of Administration

The C17-derivatized dexamethasone antiprogestins of the invention and compounds identified using any of the methods disclosed herein may be administered to subjects with a pharmaceutically acceptable diluent, carrier, or excipient, in unit dosage form. By “pharmaceutically acceptable” is meant a material that is not biologically or otherwise undesirable, i.e., the material may be administered to an individual along with a C-17-derivatized dexamethasone antiprogestin without causing any undesirable biological effects or interacting in a deleterious manner with any of the components of the pharmaceutical composition in which it is contained. Conventional pharmaceutical practice may be employed to provide suitable formulations or compositions to administer such compositions to subjects. Any appropriate route of administration may be employed, for example, but not limited to, intravenous, parenteral, transcutaneous, subcutaneous, intramuscular, intracranial, intraorbital, ophthalmic, intraventricular, intracapsular, intraspinal, intracisternal, intraperitoneal, intranasal, intrarectal, intravaginal, aerosol, or oral administration. Therapeutic formulations may be in the form of liquid solutions or suspensions; for oral administration, formulations may be in the form of tablets or capsules; for intranasal formulations, in the form of powders, nasal drops, or aerosols; for intravaginal formulations, vaginal creams, suppositories, or pessaries; for transdermal formulations, in the form of creams or distributed onto patches to be applied to the skin.

Methods well known in the art for making formulations are found in, for example, Remington: The Science and Practice of Pharmacy (19th ed.) ed. A. R. Gennaro, Mack Publishing Company, Easton, Pa. 1995. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Dosage

The C-17-derivatized dexamethasone antiprogestins of the invention may be administered to a subject in an amount sufficient to inhibit progestin activity in a subject in need thereof, or to treat, prevent, inhibit, or regulate a progestin-dependent condition in a subject in need of such treatment, prevention, inhibition, or regulation. One of ordinary skill in the art will understand that the optimal dosages used will vary according to the individual being treated, the particular compound being used, and the chosen route of administration. The optimal dosage will also vary among individuals on the basis of age, size, weight, gender, and physical condition. Methods for determining optimal dosages are described, for example, in Remington: The Science and Practice of Pharmacy (19th ed.) ed. A. R. Gennaro, Mack Publishing Company, Easton, Pa. 1995.

The amount of active ingredient that may be combined with a carrier material to produce a single dosage form will vary depending upon the disease treated, the animal species, and the particular mode of administration. A therapeutically effective amount may be determined by routine experimentation and by analogy from the amounts used to treat the same disease states with analogous antiprogestin compounds. For example, a unit dose of the antiprogestin may preferably contain between 0.001 milligram (mg) and 1 gram of the active ingredient. Typically, for treatment of humans, an antiprogestin of the invention would be administered in an amount ranging from approximately 0.001 to 10 mg/kg of body weight, and preferably, from 0.1 to 3 mg/kg would be administered. The compounds may be administered daily one to four times per day, preferably once or twice a day. Antiprogestins may also be administered weekly, monthly, or sporadically (for example, after coitus), as is well known in the art. Examples of antiprogestin administration regimens may be found, for example, in Bygdeman et al., Eur J Contracept Reprod Health Care 4:103-107, 1999 and Spitz and Robbins, Hum Reprod Update 4:584-593, 1998.

Efficacy

The efficacy of administration of a particular dose of an C-17-derivatized dexamethasone antiprogestin can be determined by evaluating the particular aspects of the medical history, signs, symptoms, and objective laboratory tests that are known to be useful in evaluating the status of a subject in need of inhibition of progestin activity or a subject that requires treatment, prevention, inhibition, or regulation of a progestin-dependent disease or condition. These signs, symptoms, and objective laboratory tests will vary, depending upon the particular disease or condition being treated or prevented, as will be known to any clinician who treats such subjects or a researcher conducting experimentation in this field. For example, if, based on a comparison with an appropriate control group and knowledge of the normal progression of disease in the general population or the particular individual: 1) a subject's frequency or severity of recurrences is shown to be improved, 2) the progression of the disease is shown to be stabilized or delayed, or 3) the need for use of other medications for treating the condition or disease is lessened or obviated, then a particular treatment will be considered efficacious.

In a specific example, in using the antiprogestins of the present invention to treat a progestin-responsive tumor, inhibition of tumor growth or metastases, or shrinkage of the tumor, are indications that an antiprogestin treatment is efficacious. Similarly, a decrease in the signs or symptoms of a diagnosed progestin-dependent disease or condition (such as endometriosis or uterine fibroids) after antiprogestin treatment indicates the efficaciousness of the treatment.

Diagnostic Uses and Experimental Uses of Antiprogestins

Progestins are involved in diverse physiological processes. Although best studied for their role in the female reproductive cycle and in diseases and conditions of the female reproductive organs such as the breast, ovary, uterus, and cervix, progestins also play a role in male animals, both in normal physiology and in disease. In particular, progestin may play a role in the development and/or progression of prostate cancer and meningiomas. As progestin levels in both male and female subjects both affect and reflect a subject's health and reproductive status, measurement of a subject's progestin and/or antiprogestin levels allows the diagnosis and/or monitoring of a progestin-dependent disease or condition in the subject. The C-17-derivatized dexamethasone antiprogestins of the invention may be employed in diagnostic assays for the measurement of progestin levels and/or antiprogestin levels in such subjects. These diagnostic assays may also be used to monitor progestin, progestin agonist, and/or antiprogestin levels in a subject receiving treatment for the purpose of inhibiting or regulating progestin activity (e.g., for contraception, regulation of menses, or induction of cervical ripening).

For example, to detect a progestin or progestin agonist in a sample from a subject (e.g., a tissue biopsy, blood, urine, cells, cell lysate, or cell extract), the sample is added to a reaction mixture (e.g., within a well of a microtiter plate) that contains a progesterone receptor. A C-17-derivatized dexamethasone antiprogestin of the invention is added to the well either before, after, or at the same time as the addition of the sample to the well. One of ordinary skill in the art will know how to measure the amount progestin or progestin agonist activity using one of the many receptor binding assays known in the art, such as the cell-free receptor binding assay described in Example V below. The relative increase in progestin activity, for example, indicated by a decrease in binding of a labeled (e.g., radioactive, colorimetric, or fluorometric) C-17-derivatized dexamethasone antiprogestin to the progesterone receptors in the sample well, as compared to a control well not containing the sample, indicates the relative level of progestin in the sample. One of ordinary skill in the art will understand that analogous assays may also be employed in high-throughput screens to identify compounds that have progestin activity by using a test compound as the sample, rather than a sample from a subject.

One of ordinary skill in the art will also understand that assays for progestin activity in a sample, or high-throughput screens to identify compounds that have progestin activity, may also be performed using one of the many known reporter gene assays, for example, the luciferase reporter gene assay described in Examples II-IV below. In these assays, the reaction mixture (e.g., in a microtiter well) would contain a progesterone receptor, a reporter gene that is transcriptionally activated by the progesterone receptor (e.g., the reporter gene described in Examples II-IV below), and any other factors necessary for transactivation of the reporter gene by the progesterone receptor. The reporter gene and progesterone receptor may be within a cell or within a cell-free extract, for example. The microtiter well would also contain a C-17-derivatized dexamethasone antiprogestin of the invention, which may be introduced into the well either before, after, or at the same time as the sample or test compound is added to the well. To measure progestin activity in a sample, or to identify a test substance as having progestin activity, the sample or test substance is added to the microtiter well, and the amount of reporter gene activity is then measured. An increase in reporter gene activity, compared to the amount of reporter gene activity in a control microtiter well lacking the sample or the test compound, indicates the progestin activity of the sample or test compound. One of skill in the art will be well aware that numerous variations on this basic reporter gene assay may be used in a similar manner.

Antiprogestin activity may be analogously detected in a sample from a subject, for example, using a progesterone receptor binding assay as described above. In one example, the sample is added to a reaction mixture containing a progesterone receptor and a progestin or progestin agonist. The progestin activity in the mixture is measured (e.g., by the amount of binding to progesterone receptors) and compared with the progestin activity in a second microtiter well that contains, in addition to a progesterone receptor and a progestin or progestin agonist, a C-17-derivatized dexamethasone antiprogestin of the invention. The progestin activity in the second microtiter well is compared to the progestin activity in a third well that contains the progesterone receptor and the progestin or progestin agonist, but lacks the antiprogestin. A decrease in progestin activity in the first well that is at least about 20% of the decrease in progestin activity of the second well (wherein the decrease in the second well is relative to the third, control well), indicates the relative amount of antiprogestin activity in the sample. Analogous assays may also be employed in high-throughput screens to identify compounds that have antiprogestin activity by using a test compound as the sample, rather than a sample from a subject.

In addition, reporter gene assays (such as the reporter gene assay described in Example V below, or one of the other numerous reporter gene assays known in the art) may be used lo measure antiprogestin activity in samples or test compounds being assayed for antiprogestin activity. In a specific example, the sample or test compound is added to a reaction mixture (e.g., in a microtiter well) containing a progesterone receptor and a reporter gene regulated by the progesterone receptor, as described above. Also added to the well is a progestin or a progestin agonist. The amount of reporter gene activity within the well is measured and compared with the amount of reporter gene activity in a second well containing a progesterone receptor, a progesterone receptor-responsive reporter gene, a progestin or a progestin agonist, plus a C-17-derivatized dexamethasone antiprogestin. A decrease in reporter gene activity in the first well that is at least about 20% of the decrease in reporter gene activity in the second well (wherein the decrease in the second well is in comparison to the amount of reporter gene activity in a third well lacking the antiprogestin) indicates that the sample or test compound contains antiprogestin activity.

The antiprogestin compounds of the invention can also be used to detect genes that are positively or negatively regulated by antiprogestins. For example, a sample containing mammalian cells or a mammalian cell-free transcription or transcription/translation extract (i.e., containing progesterone receptors, genes that are transcriptionally regulated by progesterone receptors, and any other factors necessary to support transcription or transcription/translation) is contacted with a C-17-derivatized antiprogestin of the invention, such that the antiprogestin binds to the progesterone receptors and modulates expression of one or more downstream genes (one of ordinary skill in the art will understand that it may be necessary or desirable to add exogenous progestin or a progestin agonist to the samples either before, after, or at the same time as the antiprogestin is added to the sample). The mRNA and/or protein patterns in the treated samples are compared to those in untreated control samples, and increases or decreases in specific mRNAs or proteins in the treated sample indicate an mRNA or protein encoded by a gene that is positively or negatively regulated by the particular antiprogestin used in the experiment. The gene encoding the mRNA or protein is then identified by well known methods (for example, using Serial Analysis of Gene Expression, or SAGE, as described in Velculescu et al., Science 270:484-487, 1995). Comparison of antiprogestin-regulated gene expression patterns in various types of cells or cell extracts (e.g., from liver versus ovary) provides valuable information regarding the physiological effects of a given antiprogestin upon one or more specific genes in specific cell types. This information may assist in designing various therapeutic strategies that aim to inhibit progestin activity only in selected cell types.

Test Compounds

In general, novel drugs with progestin or antiprogestin activity may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development will readily understand that methods for dereplication (e.g., taxonomic dereplication, biological dereplication, and chemical dereplication, or any combination thereof) or the elimination of replicates or repeats of materials already known for their progestin or antiprogestin activities should be employed whenever possible.

When a crude extract is found to have progestin or antiprogestin activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that mimics or antagonizes progestin. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment can be chemically modified according to methods known in the art. Compounds identified as being of therapeutic value may be subsequently analyzed using animal models for diseases or conditions in which it is desirable to regulate progestin activity, as described herein.

C-17-Derivatized Dexamethasone Antiprogestins

The C-17-derivatized dexamethasone antiprogestins used in the methods of the invention may be readily synthesized using techniques generally known to synthetic organic chemists. Suitable methods for synthesizing Dex-Mes and Dex-Ox, for example, are provided in the Examples below, and are known in the art. Dex-Ox and Dex-Mes may be further modified, for example, by replacing the oxygen of the four-membered ring of Des-Ox (FIG. 2) with: groups containing sulfur (S), nitrogen (N), or carbon (C), including cyclic ester, amide, and thioloester groups; a three-membered ring with C alone; five-through seven-membered rings lacking or containing one or two oxygen (O), S, or N groups, including cyclic ester, amide, or thiolester groups.

Guidance for the generation of various C17-derivatized dexamethasones may also be found, for example, in Rousseau et al., J Steroid Biochem 18:237-244, 1983 (e.g., see page 238, Table 1); Giesen and Beck, Horm Metabol Res 14:252-256, 1982; Rousseau et al., Nature 279:158-160, 1979 (e.g., see page 159, Table 1); Lefebvre et al., J Steroid Biochem 33:557-563, 1989 (e.g., see page 558, Tables 1 and 2); and Lee and Soliman, Science 215:989-991, 1982. Each of the aforementioned articles is herein incorporated by reference in its entirety.

In addition, C-21-derivatized dexamethasone antiprogestins may be used in the methods of the invention. Such C-21-derivatized antiprogestins may be synthesized, for example, as described in Simons et al., J Steroid Biochem 13:311-322, 1980.

C-17-derivatized dexamethasone antiprogestins that are particularly useful in the methods of the invention, for example, those having both antiprogestin and partial agonist activity, may be readily identified using methods that are well known to those of ordinary skill in the art. For example, cited hereinabove and hereinbelow are numerous references that describe in vitro and in vivo methods for evaluating the relative antiprogestin and partial agonist activity of a compound.

The present invention is more particularly described in the following examples, which are intended as illustrative only, since numerous modifications and variations therein will be apparent to those of ordinary skill in the art.

### EXAMPLE I

Materials and Methods

Unless otherwise indicated, all operations were performed at 0° C.

Chemicals, buffers, and plasmids: [17-methyl-³H]Promegestone (R5020, 85 Ci/mmol) and non-radioactive R5020 were obtained from NEN (Boston, Mass.), Dex was obtained from Sigma (St. Louis, Mo.), Dex-Ox (Pons and Simons, J Org Chem 46:3262-3264, 1981) and Dex-Mes (Simons et al., J Org Chem 45:3084-3088, 1980) were prepared as previously described. RTI-3021-020 was a generous gift from C. Edgar Cook (Research Triangle Institute, N.C.). MMTVLuc (pLTRLuc) was obtained from Gordon Hager (NIH, Bethesda, Md.). The Renilla null luciferase reporter was obtained from Promega (Madison, Wis.).

Cell culture and transfection: Monolayer cultures of COS-7 cells were grown at 37° C. with 5% CO₂ in Dulbecco's modified Eagle's medium (DMEM; GIBCO/BRL Life Technologies, Inc.) supplemented with 5% of fetal bovine serum, respectively. The 1470.2 mouse mammary adenocarcinoma cells (Pennie et al., Mol Cell Biol 15:2125-2134, 1995) were grown in DMEM with 4.5 g glucose/L (Quality Biologicals, Inc.) whereas T47D cells were grown in Dulbecco's modified Eagle's medium with high glucose (4.5 g/L, 2 mM glutamine, and 60 ng/ml insulin), both with 10% of fetal bovine serum. The 1470.2 cells were transiently transfected with the human PR-B construct, 1 μg of MMTVLuc, and 50 ng Renilla null luciferase, with the total transfected DNA brought up to 3 μg/60 mm dish with pBSK⁺DNA, using the calcium phosphate method as previously described (Szapary et al., J Biol Chem 271:30576-30582, 1996). Following 24 h of steroid treatment, the cells were harvested in 1× Passive Lysis Buffer (0.5 ml/dish; Promega, Madison, Wis.). Twenty μl of the cell lysates were used to assay for luciferase activity using the Dual-Luciferase Assay System from Promega (Madison, Wis.) and the methods recommended by the supplier. CV-1 and T47D cells were transiently transfected with human PR plasmids and 1 μg reporter construct (GREtkCAT, GREtkLuc, or MMTVLuc) as assayed as described (Szapary et al., J Biol Chem 271:30576-30582, 1996).

Assay for steroid competition: Transient transfection of COS-7 cells with 10 μg/60 mm dish with human PR-B cDNA was performed as previously described (Szapary et al., Mol Endocrinol 7:941-952, 1993). Cytosols of transfected cells containing the steroid-free receptors were obtained by the lysis of cells at −80° C. and centrifuged at 15,000×g (Simons, et al., Biochemistry 23:6876-6882, 1984). Thirty percent cytosol with 20 mM sodium molybdate was added to 2.5 nM [³H]R5020±2-, 20-, 200-, or 2000-fold excess non-radioactive competitor and incubated at 0° C. for 18 h. Unbound [³H]R5020 was removed by dextran-coated charcoal. Specific binding was calculated by subtracting the background dpms seen with 2000-fold excess R5020 from the [³H]R5020 total binding in the presence or absence of non-radioactive competitor. The specific dpms obtained with [³H]R5020+ competitor were divided by the specific dpms in the absence of competitor and expressed as percent of non-competed binding.

Steroids: FIG. 2 shows the structures of the agonist and antagonist steroids used in our studies. The structures of the agonists for GR (Dex) and PR (R5020) are shown above the bold line. The structures for the two new partial antiprogestins of the invention (Dex-Mes and Dex-Ox), which previously have been described as antiglucocorticoids (Simons and Thompson, Proc Natl Acad Sci USA 78:3541-3545, 1981; Pons and Simons, J Org Chem 46:3262-3264, 1981; Lamontagne et al., Endocrinology 114:2252-2263, 1984), are below the bold line. The previously described antiprogestins (RTI 3021-020 and RU 486), are also below the bold line.

Data analysis: The biological activity with subsaturating concentrations of agonist, or saturating concentrations of antagonist, was expressed as percent of maximal activity with saturating concentrations of agonist (30 nM R5020 unless otherwise noted). The fold induction with 30 nM R5020 was calculated as either the normalized chloramphenicol acetyl transferase (CAT) or luciferase activity with 30 nM R5020 divided by the basal activity obtained with ethanol. Individual values were generally within ±20% of the average, which was plotted.

### EXAMPLE II:

PR Partial Agonist Activity of Dex-Mes

Three different cell lines (CV-1, T47D, and 1470.2) were used to examine the biological activity of various steroids. The 1470.2 cells were selected for the majority of assays for two reasons. First, they have no endogenous PR, thereby allowing the introduction by transient transfection of either PR-A or PR-B isoforms. Second, the 1470.2 cells afforded a much larger dynamic range of transactivation vs. concentration of transiently transfected PR than did CV-1 cells (3 to 20-fold induction over about a 5-fold concentration of PR). Therefore, experiments in 1470.2 cells could be conducted under several conditions in which PR was limiting for transactivation.

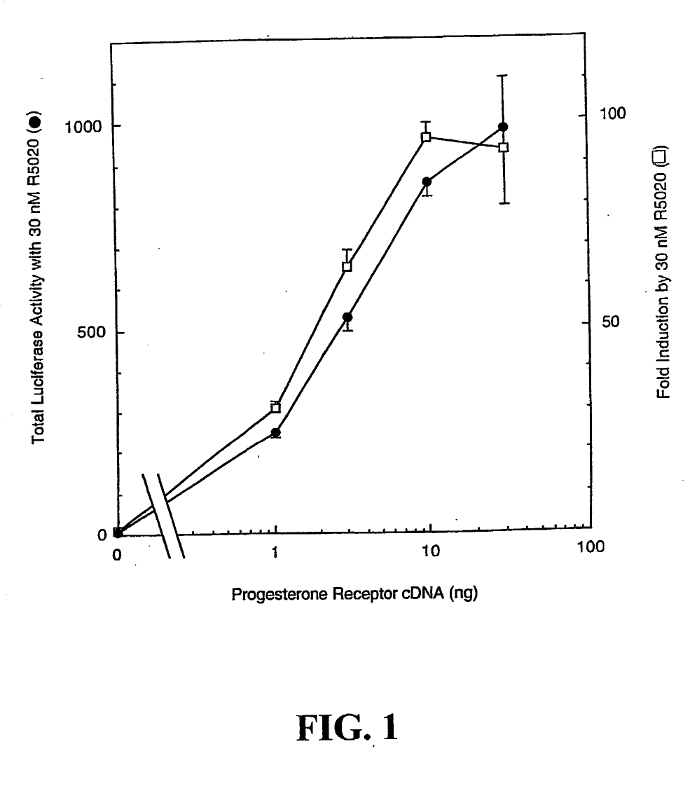

FIG. 1 is a control experiment showing the effect of PR concentration on total transactivation and -fold induction in transiently transfected 1470.2 cells. Triplicate 1470.2 cells were transfected as described in Example I above with MMTVLuc reporter and the indicated amounts of human PR-B plasmid, plus 50 ng of Renilla plasmid as an internal control for transfection, and then treated with EtOH±30 nM R5020. After determining the relative levels of Luciferase expression, the data were normalized for Renilla expression and plotted both as total activity with 30 nM R5020 (filled circles) and as fold induction above the basal (EtOH) activity (open squares) vs. PR concentration. Error bars indicate the SD of triplicate plates.

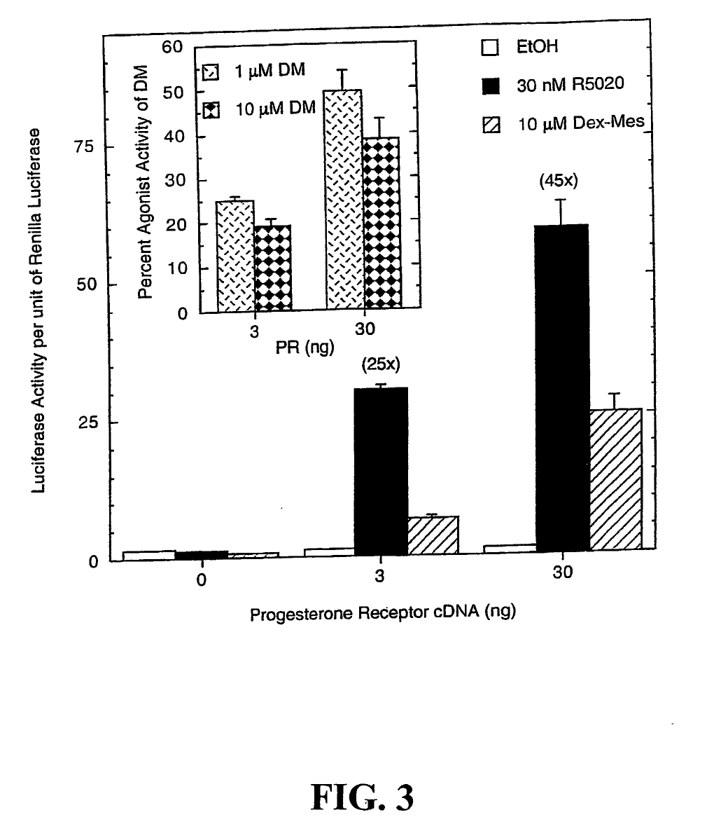

FIG. 3 shows the transactivational activity of Dex-Mes in 1470.2 cells transiently transfected with PR. Triplicate 1470.2 cells were transfected with the indicated amounts of PR-B plasmid as described in FIG. 1 and treated with EtOH±30 nM R5020, 1 μM Dex-Mes, or 10 μM Dex-Mes. All data were normalized for Renilla expression. The relative levels of total Luciferase expression were then plotted for EtOH, 30 nM R5020, and 10 μM Dex-Mes. The numbers in parentheses above the bars for 30 nM R5020 indicate the fold induction. The activity of 1 and 10 μM Dex-Mes, expressed as percent of maximal induction by 30 nM R5020, with the two PR concentrations is displayed in the insert graph. The error bars represent the SD of triplicate plates.

Transient transfection of human PR-B into 1470.2 cells permitted a good induction of the cotransfected reporter, MMTVLuc, by both R5020 and Dex-Mes (FIG. 3). When expressed as percent of the maximal activity seen with saturating concentrations of R5020, the activity with Dex-Mes did not increase in going from 1 μM to 10 μM, indicating that these were saturating concentrations of Dex-Mes. Dose-response curves yielded an EC₅₀ of about 350 nM, compared to 0.4 nM for R5020. Interestingly, the percent agonist activity of Dex-Mes did increase with added PR, just as has been observed for antiglucocorticoids with increasing amounts of GR (Szapary et al., J Biol Chem 271:30576-30582, 1996; Szapary et al., Mol Endocrinol 13:2108-2121, 1999). Nevertheless, with each PR concentration, maximal activity was reached with 1 μM Dex-Mes. Therefore, Dex-Mes displays the characteristics of a partial agonist for PR-B. The 1470.2 cells do contain GR, as determined by Scatchard analysis. However, as shown in FIG. 3, this GR was inactive with the added Dex-Mes. Dex-Mes transactivational activity was seen only after adding PR-B. In other experiments in transiently transfected CV-1 cells, Dex-Mes exhibited between 5% and 50% agonist activity for both human PR-A and human PR-B induction of either GREtkCAT or MMTVLuc reporters. Therefore, the partial agonist activity of Dex-Mes with PR was independent of receptor form (PR-A or PR-B), enhancer, promoter, gene (GREtkCAT and MMTVLuc), and cell type (1470.2 and CV-1 cells).

### EXAMPLE III:

PR Partial Agonist Activity of Dex-Ox

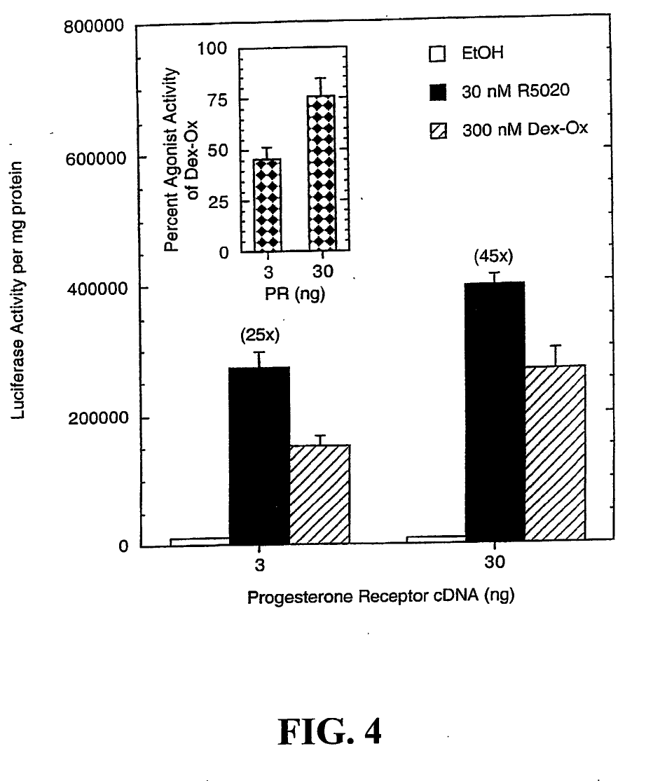

Another C-17 derivative of Dex, Dex-Ox (FIG. 2), also possessed partial agonist activity for PR induction of MMTVLuc in transiently transfected 1470.2 cells. FIG. 4 shows the transcriptional activity of Dex-Ox in 1470.2 cells with transiently transfected PR. Triplicate 1470.2 cells were transfected with the indicated amounts of PR-B plasmid as described for the experiment shown in FIG. 1, but without Renilla, and treated with EtOH±30 nM R5020, or 300 nM Dex-Ox. All data were normalized for protein levels in the cell lysates. The relative levels of total Luciferase expression were then plotted for EtOH, 30 nM R5020, and 300 nM Dex-Ox. The numbers in parentheses above the bars for 30 nM R5020 indicate the fold induction. The percent of maximal induction by 30 nM R5020 that was achieved with 300 nM Dex-Ox with the two PR concentrations is displayed in the insert graph. The error bars represent the SD of triplicate plates.

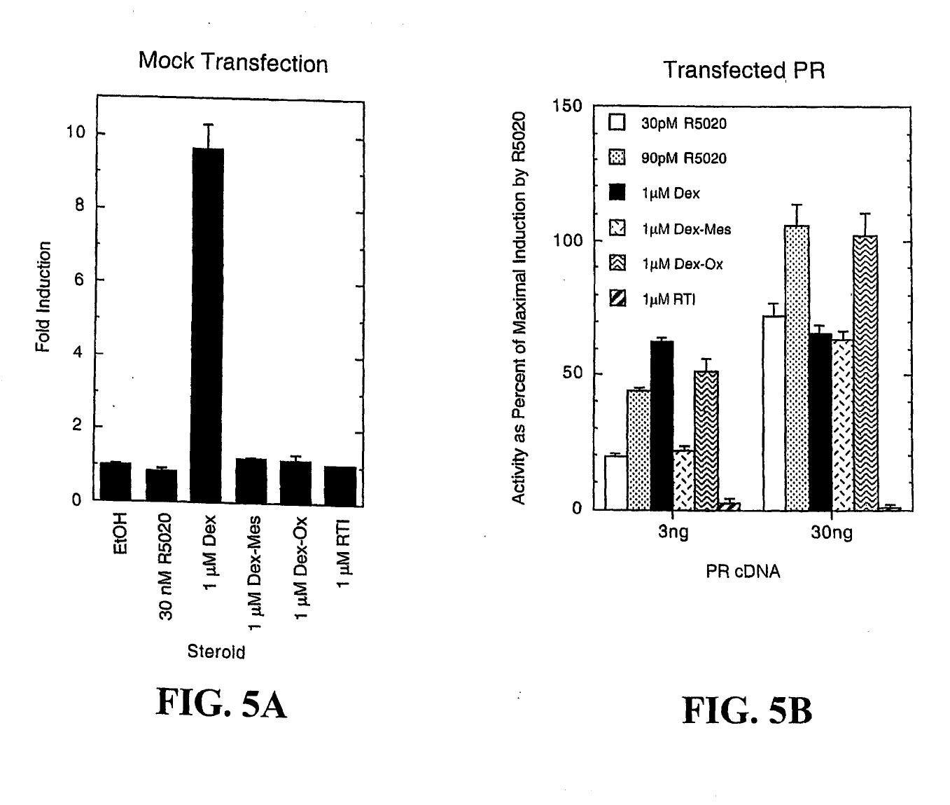

FIG. 4 shows that Dex-Ox is a partial agonist, as increasing concentrations of Dex-Ox up to 3 μM did not afford any increase in total transactivation. Dose-response curves yielded an EC₅₀ of about 30 nM, compared to 0.4 nM for R5020. Furthermore, as with Dex-Mes, the amount of agonist activity as percent of maximal transactivation by saturating concentrations of R5020 was higher in the presence of more PR. Again, the activity of Dex-Ox was not due to the endogenous GR as shown by the inactivity in the absence of transfected PR (FIG. 5A) and by the increased total activity with added PR-B (FIG. 5B). Dex-Ox also displayed between 40% and 60% agonist activity with PR-B in CV-1 cells with GREtkCAT and in T47D cells, which contain endogenous PR-A and B, with the GREtkLuc reporter. Therefore, as with Dex-Mes, the partial agonist activity of Dex-Ox with PR is independent of enhancer, promoter, gene, and cell type.

### EXAMPLE IV:

PR Agonist Activity of Dex-Mes and Dex-Ox Compared to Other Antiprogestins

The partial agonist RTI 3021-020 has been described as being inactive in CV-1 cells (Wagner et al., Proc Natl Acad Sci USA 93:8739-8744, 1996), and we have confirmed this. Furthermore, we found that Dex-Mes and Dex-Ox display much more agonist activity than either RIT 3021-020 or RU 486 in CV-1 cells. A more detailed study was then performed in 1470.2 cells, in which greater induction responses with PR can be observed.

FIGS. 5A and 5B show a comparison of relative partial progestin activity of Dex, Dex-Mes, Dex-Ox, and RTI 3021-020 in 1470.2 cells without (FIG. 5A) or with (FIG. 5B) transfected PR. Triplicate 1470.2 cells were transfected with the 3 or 30 ng of PR-B plasmid, as described for the experiment shown in FIG. 1, and treated with EtOH±R5020 (30 pM, 90 pM, or 30 nM), or 1 μM Dex, Dex-Mes (DM), Dex-Ox, or RTI 3021-020 (RTI). All data were normalized for Renilla expression. The data in cells without added PR (FIG. 5A) were expressed as -fold induction above basal levels. In cells with added PR (FIG. 5B), the activity of each steroid was plotted as percent of maximal activity with 30 nM R5020 with each concentration of PR-B. The error bars represent the SD of triplicate plates.

As shown in FIG. 5A , the only steroid with any activity in the absence of transfected PR was Dex. This observation was consistent with the results of FIGS. 3 and 4, since 1470.2 cells are devoid of PR but do contain GR. Furthermore, Dex-Mes (Szapary et al., J Biol Chem 271:30576-30582, 1996; Simons and Thompson, Proc Natl Acad Sci USA 78:3541-3545, 1981) and Dex-Ox (Lamontagne et al., Endocrinology 114:2252-2263, 1984) can display little or no agonist activity with GR. The data of FIG. 5A therefore confirm that none of the antiprogestins examined has any activity with the endogenous GR. With added PR, Dex-Mes and Dex-Ox displayed significant amounts of partial progestin activity, whereas the antiprogestin RTI 3021-020 was inactive. Thus, Dex-Mes and Dex-Ox appear to have broader-range mixed agonist activity for PR than any other antiprogestins yet described.

### EXAMPLE V:

Cell-Free Binding of Dex-Mes and Dex-Ox to PR

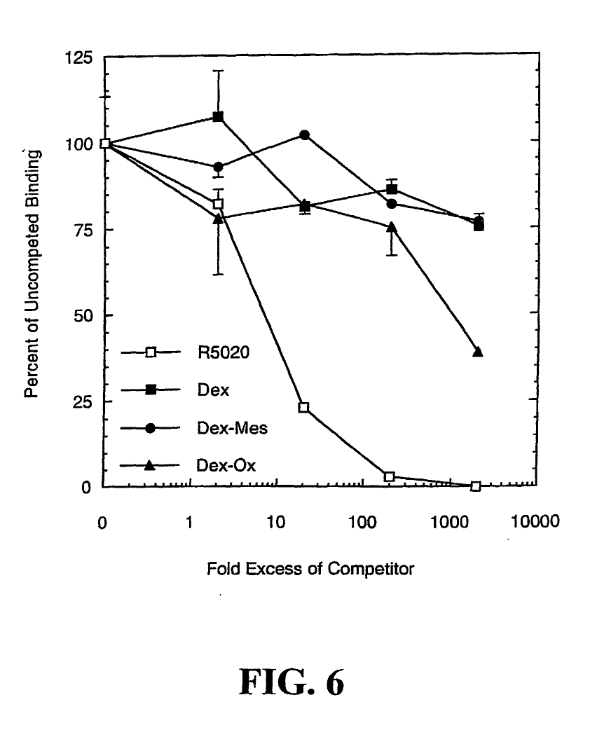

To establish that Dex-Mes and Dex-Ox are true mixed agonists for PR, we asked whether each steroid was capable of binding to PR with high affinity. FIG. 6 shows a cell-free competition of [³H]R5020 binding by non-radioactive steroids. Duplicate samples of overexpressed human PR-B containing [³H]R5020 and increasing concentrations of the indicated non-radioactive steroids were incubated and analyzed as described in Example I above. The observed residual binding data were then expressed as percent of uncompeted [³H]R5020 binding and plotted as a function of the concentration of non-radioactive steroid. The error bars indicate the range of duplicate samples.

The cell-free competition binding experiments shown in FIG. 6 indicated a binding affinity for Dex-Ox and Dex-Mes that was 1.4% and <0.3%, respectively, that of R5020. These affinities closely correlated with the EC₅₀s of Dex-Ox (1.3%) and Dex-Mes (0.11%) relative to R5020. This correlation strongly supports the conclusion that the partial agonist activity of Dex-Ox and Dex-Mes derives from their binding to PR.

### EXAMPLE VI:

Antiprogestin Activity of Dex-Mes and Dex-Ox

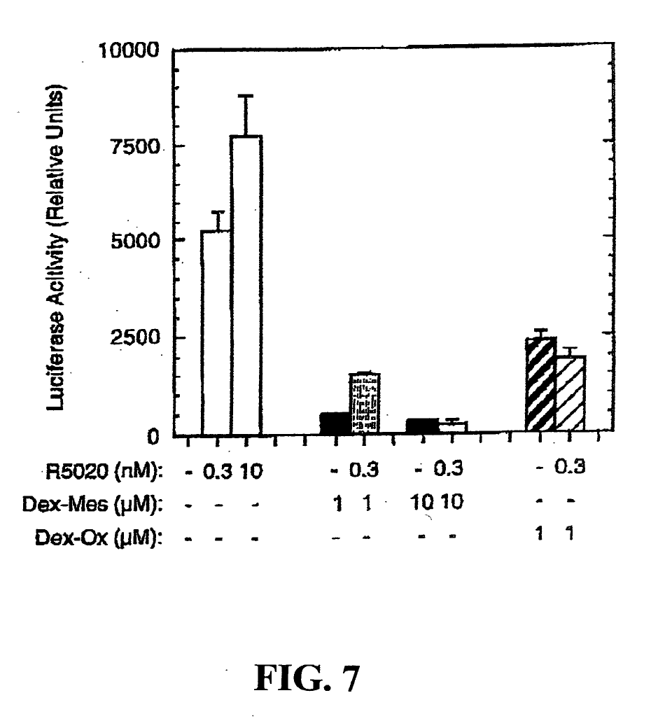

To determine whether Dex-Mes and Dex-Ox have antiprogestin activity, we assessed the ability of these steroids to antagonize progestin-activated induction of a reporter gene by PR. Triplicate cultures were transfected as described for the experiment shown in FIG. 1 and induced with the indicated concentrations of R5020±Dex-Mes or Dex-Ox. The absolute levels of luciferase activity (±S.D. of triplicate values) were plotted. In view of the lower affinity of Dex-Mes vs. Dex-Ox for PR, we used 1 μM and 10 μM Dex-Mes, but only 1 μM Dex-Ox, to inhibit the action of 300 pM R5020. With high enough concentrations, each Dex derivative reduced the activity of 300 pM R5020 to that of the competing steroid alone (FIG. 7). Therefore, Dex-Mes and Dex-Ox are antiprogestins with potencies that parallel their affinity for PR.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

What is claimed is:

1. A method of inhibiting progestin activity or of treating or preventing a progestin-dependent condition in a subject in need of such inhibition, treatment, or prevention, comprising administering to said subject a C-17-derivatized dexamethasone in an amount effective to inhibit progestin activity or to treat or prevent a progestin-dependent condition in said subject.

2. The method of claim 1, wherein said C-17-derivatized dexamethasone antiprogestin is dexamethasone-21-mesylate.

3. The method of claim 1, wherein said C-17-derivatized dexamethasone antiprogestin is dexamethasone-oxetanone.

4. The method of claim 1, wherein said subject is a mammal.

5. The method of claim 1, wherein said method is for regulating menses.

6. The method of claim 1, wherein said method is for treating or preventing a benign progestin-dependent condition.

7. The method of claim 1, wherein said method is for treating a progestin-responsive tumor.

8. The method of claim 1, wherein said method is for preventing pregnancy.

9. The method of claim 1, wherein said administering inhibits ovulation in said subject.

10. The method of claim 1, wherein said method is for inducing cervical ripening in a female.

11. The method of claim 1, wherein said administering is carried out in order to induce expulsion of an embryo or fetus from said subject.

12. A method of detecting a progestin or a progestin agonist in a sample, comprising:
a) contacting said sample with a reaction mixture comprising a progesterone receptor and a C-17-derivatized dexamethasone antiprogestin; and
b) measuring progestin activity in said reaction mixture, wherein an increase in progestin activity, as compared to the amount of progestin activity measured in a reaction mixture not contacted with said sample, detects a progestin or a progestin agonist in said sample.

13. The method of claim 12, wherein said C-17-derivatized dexamethasone antiprogestin is dexamethasone-21-mesylate.

14. The method of claim 12, wherein said C-17-derivatized dexamethasone antiprogestin is dexamethasone-oxetanone.

15. A method of detecting an antiprogestin in a sample, comprising:
a) contacting said sample with a first reaction mixture comprising a progesterone receptor and a progestin or a progestin agonist;
b) measuring progestin activity in said first reaction mixture; and
c) comparing said progestin activity in said first reaction mixture with an amount of progestin activity in a second reaction mixture comprising a progesterone receptor, a progestin or a progestin agonist, and a C17-derivatized dexamethasone antiprogestin, wherein a decrease in progestin activity in said first reaction mixture that is at least about 20% of the decrease in progestin activity in said second reaction mixture, compared to the amount of progestin activity in said second reaction mixture in the absence of said antiprogestin, detects an antiprogestin in said sample.

16. The method of claim 15, wherein said C-17-derivatized dexamethasone antiprogestin is dexamethasone-21-mesylate.

17. The method of claim 15, wherein said C-17-derivatized dexamethasone antiprogestin is dexamethasone-oxetanone.

18. A method of inhibiting progestin activity in a cell in need of such inhibition, comprising administering an effective amount of a C-17-derivatized dexamethasone antiprogestin to said cell.

19. The method of claim 18, wherein said C17-derivatized dexamethasone antiprogestin is dexamethasone-21-mesylate.

20. The method of claim 18, wherein said C17-derivatized dexamethasone antiprogestin is dexamethasone-oxetanone.

21. A method of detecting a gene whose expression level is modulated by an antiprogestin, said method comprising:
a) contacting a progesterone receptor with a C-17-derivatized dexamethasone antiprogestin, wherein said progesterone receptor is present within a sample that comprises a gene that is positively or negatively regulated by said progesterone receptor;
b) measuring the expression level of said gene in said sample; and
c) comparing said expression level of said gene in said sample with the expression level of said gene in a sample not contacted with said C17-derivatized dexamethasone antiprogestin, wherein an increase or decrease in said expression level of said gene in said sample contacted with said C-17-derivatized dexamethasone antiprogestin, compared to the expression level of said gene in said sample not contacted with said C-17-derivatized dexamethasone antiprogestin, detects a gene whose expression is modulated by an antiprogestin.

22. The method of claim 21, wherein said C17-derivatized dexamethasone antiprogestin is dexamethasone-21-mesylate.

23. The method of claim 21, wherein said C17-derivatized dexamethasone antiprogestin is dexamethasone-oxetanone.

24. The method of claim 21, wherein a progestin or progestin agonist is added to said sample prior to said measuring.

25. The method of claim 21, wherein said sample is a cell.

26. The method of claim 21, wherein said sample is a cell extract.

27. The method of claim 21, wherein said sample is a mammal.
